(19) United States
(12) Patent Application Publication
(10) Pub. No.: US 2026/0115115 A1
(43) Pub. Date: Apr. 30, 2026

(54) TYROTHRICIN FOR USE IN THE TREATMENT OR PROPHYLAXIS OF BODY ODOUR AND PREPARATIONS THEREFOR

(71) Applicant: Thomas LEIBBRAND, Heilbronn (DE)

(72) Inventor: Thomas LEIBBRAND, Heilbronn (DE)

(21) Appl. No.: 18/984,115

(22) Filed: Dec. 17, 2024

Related U.S. Application Data
(62) Division of application No. 16/632,700, now abandoned.

(30) Foreign Application Priority Data
Jul. 20, 2017 (DE) 102017116407.3

(51) Int. Cl. A61Q 15/00; A61K 8/42; A61K 8/64; A61K 8/99

(52) U.S. Cl. CPC A61K 8/42; A61K 8/64; A61K 8/99; A61Q 15/00

(57) ABSTRACT

What is described is the therapeutic or nontherapeutic use of tyrothricin for treatment and/or prophylaxis of human body odor, preferably by local (topical), superficial application and/or preferably by application to the wound-free skin and/or mucosa. Further described is a therapeutic, preferably pharmaceutical, or nontherapeutic, preferably cosmetic, composition, comprising tyrothricin and a keratolytic, for treatment and/or prophylaxis of human body odor. Likewise described are a kit comprising tyrothricin and a keratolytic, and a footwear article comprising tyrothricin.

This application is a divisional of co-pending U.S. patent application Ser. No. 16/632,700, filed Jan. 21, 2020, which is a § 371 national stage entry of International Application No. PCT/EP2018/067991, filed on Jul. 3, 2018, which claims priority to German Patent Application No. 10 2017 116 407.3, filed on Jul. 20, 2017, the entirety contents of which are incorporated herein by reference.

The present invention relates to the therapeutic or nontherapeutic use of tyrothricin for treatment and/or prophylaxis of human body odor, preferably by local (topical), superficial application and/or preferably by application to the wound-free skin and/or mucosa. The invention further relates to a therapeutic, preferably pharmaceutical, or nontherapeutic, preferably cosmetic, composition, comprising tyrothricin and a keratolytic, for treatment and/or prophylaxis of human body odor. The invention likewise relates to a kit comprising tyrothricin and a keratolytic, and also to a footwear article comprising tyrothricin.

Body odor, especially in the form of unpleasant-smelling human body odor, may emanate in particular from the surface of the skin, especially from the foot or the armpit or from poorly ventilated skin folds (“intertriginous regions”), for instance in the groin area (inguinal) or under the breast area (submammary), or else may emanate from mucosa, for instance the oral mucosa or the vaginal mucosa. Body odor of the aforementioned type or origin that is perceived as unpleasant, according to current knowledge, is generally caused and/or promoted and/or triggered by bacterial colonization and/or certain bacterial metabolism products.

In the case of origination from the surface of the skin, body odor perceived as unpleasant is favored or promoted particularly by perspiration, especially by increased or excessive perspiration. It is apparently the case that metabolism products of some microorganisms that colonize the surface of the skin, particularly bacteria that break down skin constituents and other substances released with perspiration, play an important role in the formation of this body odor. It also seems that the exact composition and amount of the bacterial colonization of parts of the skin can influence the type and extent of odor evolution. By contrast, only minor odor, if any, emanates from freshly formed, undecomposed perspiration.

Unwanted odor formation at the surface of the skin is favored or promoted by influencing factors such as severe to excessive perspiration and poorly ventilated clothing. Excessive sweating is also known by the term “hyperhidrosis”, especially by the term “primary focal hyperhidrosis”. Excessive perspiration in the foot region is also known by the terms “Hyperhidrosis plantaris” and “Hyperhydrosis pedis/pedum”. Excessive sweating in the armpit region is also known by the term “Hyperhidrosis axillaris”.

Excessive perspiration is often associated with a phenomenon known as “bromhidrosis”: bromhidrosis refers to malodorous perspiration and is a specific type of hyperhidrosis where the perspiration produced to an enhanced degree constantly soaks the horny layer of the skin and promotes the propagation of the germ flora present therein. The degradation of the keratin in the horny layer gives rise to degradation products, for example short-chain fatty acids and amines, which can be further metabolized by particular germs in said germ flora to give various secondary products. Some of these germs seem to have a multitude of metabolic pathways, some of which are unusual with regard to the occurrence of particular metabolic products: one peculiarity is, for example, the synthesis of mycothiol rather than glutathione which is observed in the case of some microorganisms. Moreover, volatile (in particular short-chain) organic thiols also seem to form as metabolic products of some of the germs in said germ flora. Volatile organic thiols often have an odor perceived by humans as being particularly unpleasant, in some cases as disgusting, and can therefore make a considerable contribution to a body odor perceived to be unpleasant-particularly in the region of the groin region, the armpits and the feet, for instance the spaces between the toes.

Apocrine sweat or sebum that originates from the apocrine glands is degraded by bacteria, which leads to significant odor that can vary according to the composition of the degradation or metabolism products. “Apocrine secretion” is generally understood to mean the process when secretory vesicles with surrounding apical cytoplasm are pinched off from the gland cell by part of the cell membrane.

The phenomenon of bromhidrosis can be even further differentiated into the sub-categories of “apocrine bromhidrosis” and “eccrine bromhidrosis”. Eccrine glands are glands that release their secretion to a clear surface (for example the skin or the digestive tract), with formation of secretion without loss of cytoplasm identifiable by light microscopy on secretion (e.g. small perspiration glands).

Apocrine bromhidrosis refers to marked secretion of perspiration, predominantly by apocrine axillary perspiration glands. Organic constituents of apocrine perspiration are then broken down by bacteria to give unpleasant-smelling metabolism products. Inadequate hygiene promotes the occurrence of apocrine bromhidrosis. It occurs predominantly after sexual maturity.

Eccrine bromhidrosis refers to unpleasant odor resulting from secretion of eccrine perspiration with different causes. The subcategory of keratogenic eccrine bromhidrosis refers to a state where hyperhidrosis, up to and including excessive hyperhidrosis, leads to softening of the uppermost skin layer (uppermost layer of the epidermis; Stratum corneum), which is then broken down by bacteria, with subsequent odor formation. This phenomenon is also known as “bromhyperhidrosis”. Keratogenic eccrine bromhidrosis occurs particularly among young and middle-aged adults, occasionally also in children, especially male children. The particular regions affected are the regions of the soles of the feet (plantary), the surfaces of the hand (palmary) and intertriginous regions.

One cause of body odor perceived as being unpleasant that emanates from the feet is poorly ventilated footwear. For instance, after prolonged wearing of tight and poorly ventilated shoes, on the ridged skin which is swollen up by the accumulated moisture and partly macerated—particularly in the more highly stressed parts of the skin—there are often pit-shaped lesions of the horny skin which, when enlarged under the microscope, look like superficial pitting or have a punched-out and crater-like appearance. These skin lesions are also referred to as “pits”. These findings are also known as ‘Keratoma sulcatum’, “pitted keratolysis” or “plantar pitting”. The wearing-away of the superficial horny skin layers, for example by means of standard scraping, can temporarily prevent, but generally not entirely eliminate, unpleasant body odor emanating from the foot. The above-described pits always recur.

The people affected can perceive their own body odor that has arisen in the above manner to be highly oppressive, extending as far as psychological problems. Even though affected people often wash several times per day, the idea of “smelling unpleasant” remains, which in extreme cases can grow to become deluded perceptions of odor as a particular form of dysmorphia.

Various means and methods of treatment of body odor perceived as unpleasant, particularly of body odor emanating from the foot or the armpit have already been proposed. As well as the improvement of hygiene and the use of well-ventilated clothing, these include antibacterial soaps and lotions for washing of the body parts affected (often containing, for example, the active ingredient triclosan); regular foot baths with tea tree oil, for example; deodorants and antiperspirants, for example containing aluminum salts such as aluminum trichloride; use of usually sweat-drying foot creams or powders, for example containing zinc oxide, zinc and/or fragrances; shoe deodorants or foot sprays that contain fragrances; insoles modified, for example, with drying agents such as zinc oxide, sodium carbonate, activated carbon and/or cedar wood; the sterilization of shoes by means of UV rays; the wearing of natural wool socks, for example merino wool socks, and/or the use of particular food supplements, for example zinc.

But these known means and methods are unsuitable for various reasons for fast-acting, long-lasting and/or a long-term treatment and/or prophylaxis of body odor. In most of the aforementioned cases, regular, usually daily, application or treatment is necessary. In some of the aforementioned cases, the unpleasant odor is merely masked without eliminating the cause thereof, at least for a prolonged period.

There is accordingly a need for a composition for rapid- and long-acting treatment of human body odor, preferably of body odor emanating from the surface of the skin. An aforementioned composition should also preferably not cause or promote any antibiotic resistances and be well-tolerated.

Tyrothricin (CAS RN: 1404-88-2; cf. also definition and description in the European Pharmacopoeia, for example according to entry 01/2005:1662) is a mixture of various antibacterially and partly antimycotically active linear and cyclic polypeptides from the groups of the gramicidines and tyrocidines; in addition, further structurally related polypeptides occur in small amounts. Tyrothricin can be obtained from the fermentation broth of “Brevibacillus brevis Dubos”. It is assigned to the polypeptide antibiotics, which also include actinomycin, bacitracin and the polymyxins. In accordance with its antibacterial mode of action, tyrothricin irreversibly disrupts the cell membrane of various microorganisms. In that way, it is similar to the antimicrobial peptides (also referred to as “host defense peptides”), as known from eukaryotes, for example defensin and cathelicidin. In spite of decades of use of tyrothricin, no evolutions of resistance have as yet become known as in the case of other antibiotics.

Tyrothricin is already being used as a locally active antibiotic in the form of throat tablets in the event of a sore throat and throat pain with difficulty in swallowing, in the event of inflammation of the pharynx and larynx, and in the event of inflammation of the oral mucosa and the gums.

Likewise already known is the use of tyrothricin for alleviating treatment of small-area, superficial skin wounds with bacterial superinfection with a minor degree of weeping, for example lacerations, scratches or grazes.

An overview of known uses of tyrothricin can be found, for example, in C. Lang et al. in Pharmazie 71 (2016) 299-305.

Document DE 2316597 describes a dermatological formulation that enables enhanced penetration of various pharmacologically active substances, including tyrothricin, as antimicrobial composition, for example for superficial treatment of burns, into and through the tissue or skin. The formulation described contains sugar esters or sugar ethers in combination with a sulfoxide or a phosphine oxide.

Document DE 36 04 865 A1 describes a skin cosmetic that contains tyrothricin inter alia and is suitable for treatment in the case of superinfectious dermatoses.

Document EP 0 355 536 A2 describes a flexible hydrophilic gel film and a method for production and use thereof. Tyrothricin is cited as one of the use examples of buccal administration forms of the gel film.

Document WO 99/061011 describes the use of tyrothricin for treatment of certain viral infections, including on the treatment of molluscum contagiosum on the skin.

The German Patent and Trademark Office found the following prior art for the priority application of the present application:
Knols B. G. J.: On human odor, malaria mosquitoes, and Limburger cheese. In: The Lancet., Vol. 348, 1996 Nov. 9, 1322.

A primary object of the present invention was that of providing a therapeutic or nontherapeutic composition for use in the treatment and/or prophylaxis of human body odor, preferably of human body odor emanating from the surface of the skin, which has additional advantages over the prior art.

It was a further object of the invention to provide a therapeutic or nontherapeutic, preferably cosmetic, composition suitable for use in the treatment and/or prophylaxis of human body odor, especially of human body odor emanating from the surface of the skin. It was also a specific further object of the invention to provide such aforementioned therapeutic or nontherapeutic, preferably cosmetic, compositions having only low intrinsic odor, if any.

It was a further object of the invention to provide a kit suitable for use in the treatment and/or prophylaxis of human body odor, especially of human body odor emanating from the surface of the skin.

The invention, and combinations of preferred parameters, properties and/or constituents of the present invention that are preferred in accordance with the invention, are specified and/or defined in detail in the appended claims.

Specific and/or preferred embodiments of the invention and the inventive combinations thereof are described more specifically hereinafter. Unless stated otherwise, preferred aspects, parameters or embodiments of the invention can be combined with other aspects, parameters or embodiments of the invention, especially with other preferred aspects, parameters or embodiments. The combination of respectively preferred aspects, parameters or embodiments with one another results in turn in respectively preferred aspects, parameters or embodiments of the invention, unless stated otherwise.

Embodiments, aspects, parameters or properties that are described or described as preferred in connection with the present invention for the inventive use or application of tyrothricin are each also applicable correspondingly or mutatis mutandis to the composition of the invention or use or application thereof, to the kit of the invention and use thereof, and to the footwear article of the invention.

Where a description is given hereinafter of a novel use or application of tyrothricin, of a tyrothricin-containing composition of the invention or the use or application thereof, of a kit of the invention or use thereof, of a method of the invention and of a footwear article of the invention that “comprise” or “contain” embodiments, constituents or features specified in detail, the corresponding definition to be understood in a narrower context shall also be disclosed in each case, in which said use or application of tyrothricin, the tyrothricin-containing composition of the invention or use or application thereof, the kit of the invention or use thereof, the method of the invention and the footwear article of the invention “consist” of these embodiments, constituents or features that have each been specified in detail.

It has now been found that the primary object and further objects and/or partial objects of the present invention are achieved by the therapeutic, preferably pharmaceutical, or nontherapeutic, preferably cosmetic, use of tyrothricin and/or a tyrothricin-comprising composition of the invention for treatment and/or prophylaxis of human body odor.

Preference is given to an aforementioned inventive use of tyrothricin, wherein the body odor is caused and/or promoted and/or triggered by bacterial colonization and/or bacterial metabolism products and/or perspiration and/or wherein the body odor is associated with one or more of the aforementioned phenomena.

Also preferred is a use of the invention or a preferred use of the invention in which the body odor emanates from the surface of the skin, preferably from the foot and/or the armpit and/or an intertriginous region and/or wherein the body odor emanates from a mucosa, preferably the oral mucosa and/or the vaginal mucosa. Intertriginous regions here preferably include poorly ventilated regions of the surface of the skin in the groin (inguinal) and/or under the breast region (submammary), preferably under the female breast region, and/or the spaces between the toes.

Preference is likewise given to a use of the invention or use of the invention specified above as preferred, wherein the body odor emanates from the surface of the skin, preferably from the foot and/or the armpit and/or an intertriginous region, more preferably from the foot, including the intertriginous regions of the foot.

If the body odor emanates from the surface of the skin, preferably from the foot and/or the armpit and/or an intertriginous region, particular preference is given to the inventive use of tyrothricin in the event of excessive perspiration, preferably in the presence of excessive perspiration that can be attributed to a phenomenon selected from the group consisting of hyperhidrosis, preferably primary focal hyperhidrosis, Hyperhidrosis plantaris, Hyperhidrosis axillaris and/or Hyperhidrosis pedis; Keratoma sulcatum (also referred to as “plantar pitting” or “pitted keratolysis”, see above) and bromhidrosis, preferably apocrine bromhidrosis, eccrine bromhidrosis and/or keratogenic eccrine bromhidrosis; and mixed forms of the aforementioned phenomena.

Preference is also given to a use of the invention or use of the invention specified above as preferred, wherein the application is local and superficial and/or wherein the application is to the wound-free skin and/or to the wound-free mucosa, preferably to the wound-free skin.

“Wound-free skin” in the context of the present invention means that the skin is preferably uninjured and especially does not have any surface wounds, for example lacerations, scratches or grazes. By contrast, warts, hornification and/or onychomycosis, in the context of the present invention, are not covered by the term “wound-free skin”. Correspondingly and mutatis mutandis, “wound-free mucosa” in the context of the present invention means that the mucosa is preferably uninjured. By way of clarification, it should be emphasized that relatively small surface wounds to the skin, such as relatively small lacerations, scratches or grazes, from a purely medical point of view, are of no concern in respect of the therapeutic or nontherapeutic use of tyrothricin for treatment and/or prophylaxis of human body odor and do not constitute any hindrance or contraindication for an aforementioned use or application of the invention.

A “local superficial use or application” in the context of the present invention means that the tyrothricin active ingredient preferably acts in a non-percutaneous and/or non-systemic and/or non-transdermal manner, i.e. not through uptake via the skin into the body, for example not into the bloodstream or lymph circulation, i.e. not through resorption or absorption of tyrothricin through the skin and/or through the mucosa. However, an inventive local and/or superficial application or use shall include the penetration of tyrothricin into the uppermost skin layer or epidermal layer (Stratum corneum), and possibly even into skin layers beneath.

In in-house studies, it has been found that, in the case of local superficial application, at least once daily, of a tyrothricin-containing composition over a period (“treatment period”) in the range from 1 to 7 days, for instance in the range from 2 to 7 days, such as in the range from 3 to 7 days and generally in the range from 5 to 6 days, body odor perceived as unpleasant is significantly reduced and surprisingly often even permanently eliminated (“successful application”).

Moreover, it has been found in in-house studies that said significant reduction or even elimination of the body odor perceived as unpleasant, in a significant number of the cases examined, typically after the end of application of the tyrothricin-containing composition, lasts over a longer period (“period of success”) in the range from 7 to 14 days, in many cases even in the range from 7 to 21 days, in a significant number of cases even in the range from 7 to 28 days and in some cases even in the range from 7 to 30 days. Repetition of the application with a tyrothricin-containing composition was thus indicated again only after the period of success had elapsed, in most cases after 7 days had elapsed after conclusion of the or a preceding treatment period, in the multitude of cases after 14 days had elapsed after conclusion of the or a preceding treatment period, in many cases after 21 days had elapsed after conclusion of the or a preceding treatment period, and in some cases after 28 days had elapsed after conclusion of the or a preceding treatment period.

The present invention also relates to tyrothricin for use in the treatment and/or prophylaxis of human body odor. Tyrothricin here is preferably a constituent of a composition of the invention as described hereinafter.

All the configurations and combinations of the invention that have been specified above for the inventive use of tyrothricin, including the configurations and combinations specified as preferred in each case, are also applicable without restriction and if appropriate mutatis mutandis in respect of tyrothricin for the above-specified application of the invention.

The present invention also relates to a method of therapeutic or nontherapeutic, preferably cosmetic, treatment and/or prophylaxis of human body odor, wherein tyrothricin or a tyrothricin-containing composition, preferably a composition of the invention as described hereinafter, is applied locally and superficially to the preferably wound-free skin and/or to the preferably wound-free mucosa. The aforementioned application is preferably to the preferably wound-free skin. The aforementioned application or preferred application is preferably in a therapeutically or nontherapeutically, preferably cosmetically, active dose.

All the configurations and combinations of the invention that have been specified above for the inventive use of tyrothricin, including the configurations and combinations specified as preferred in each case, are also applicable without restriction and if appropriate mutatis mutandis in respect of the above-specified method of the invention.

An above-specified method of the invention and/or a use of the invention and/or an application of the invention of tyrothricin and/or a tyrothricin-containing, preferably inventive, composition (preferably as described hereinafter), in each case for treatment and/or prophylaxis of human body odor, is suitable in principle for daily application, preferably for once- or twice-daily application, even over prolonged periods.

Owing to the advantageous properties of the method of the invention and/or of the use of the invention and/or of the inventive application of tyrothricin and/or a tyrothricin-containing, preferably inventive, composition-especially owing to the rapid- and long-acting treatment or prophylaxis of human body odor—only less frequent application is required in many cases.

Preference is given to an above-specified method of the invention and/or to a use of the invention and/or to an inventive application of tyrothricin and/or a tyrothricin-containing, preferably inventive, composition (preferably as described hereinafter), in each case for treatment and/or prophylaxis of human body odor, wherein tyrothricin and/or a preferably inventive tyrothricin-containing composition (preferably as described hereinafter),
for a treatment period or a first treatment period, preferably in the range from 1 to 7 days, preferably in the range from 2 to 7 days, more preferably in the range from 3 to 7 days and most preferably in the range from 5 to 6 days,
is applied locally and superficially at least once daily, preferably twice daily, more preferably twice daily, once in the morning and once in the evening,
   wherein the treatment period is preferably not repeated more frequently than every 7 days, more preferably not more frequently than every 14 days,
   and/or
   wherein the application is preferably to the wound-free skin and/or the wound-free mucosa.

In a preferred embodiment of the above-specified preferred method of the invention and/or of the above-specified preferred use of the invention and/or of the above-specified preferred application of the invention, tyrothricin and/or a preferably inventive tyrothricin-containing composition, after a first treatment period (as defined above or as defined above as preferred),
for a second or further treatment period (“recurrent prophylaxis”), preferably in the range from 1 to 4 days, more preferably in the range from 2 to 3 days,
is applied locally and superficially at least once daily, preferably twice daily, more preferably twice daily, once in the morning and once in the evening,
   wherein the second or further treatment period is preferably not repeated more frequently than every 30 days, more preferably not more frequently than every 21 days, most preferably not more frequently than every 14 days, in each case calculated from the end of the first or preceding treatment period,
   and/or
   wherein the application is preferably to the wound-free skin and/or the wound-free mucosa.

Preferably, in the context of the above-specified use and/or application of the invention and/or of the above-specified method of the invention, tyrothricin or a tyrothricin-containing composition may be applied locally and superficially in customary administration forms. Examples of suitable media for application preferably to the wound-free surface of the skin are the following that are known per se: creams, gels, lotions, powders, powder sprays, roll-on formulations, ointments, foams, sprays, sticks and tinctures. Examples of suitable media for application to the preferably wound-free mucosa—in the case of the oral mucosa—are the following that are known per se: bonbons, pastilles, lozenges, sublingual tablets, buccal tablets, sugar-coated tablets or mouthwashes or gargles. Examples of suitable media for application to the preferably wound-free mucosa—in the case of the vaginal mucosa—are the following that are known per se: creams, gels, lotions, powders, powder sprays, ointments, foams, sprays, tinctures, rinse solutions or suppositories.

The present invention also further relates to a therapeutic, preferably pharmaceutical, or nontherapeutic, preferably cosmetic, composition comprising tyrothricin, preferably in an antimicrobially effective amount, and at least one keratolytic, preferably in a keratolytically effective amount, preferably for use in the treatment and/or prophylaxis of human body odor. Preferred compositions of the invention are odor-neutral. By contrast, many prior art compositions for treatment and/or prophylaxis of human body odor—for instance deodorants and antiperspirants—have a greater or lesser intrinsic odor.

The composition of the invention is suitable and intended for use in the context of the inventive use of tyrothricin or in the context of the inventive aspect of “tyrothricin for use in the treatment and/or prophylaxis of human body odor”, preferably in the treatment and/or prophylaxis of human body odor, wherein the body odor emanates from the surface of the skin, preferably from the foot and/or the armpit and/or an intertriginous region.

Without any guarantee of correctness, it is assumed that the effect of the keratolytic is that at least the uppermost skin layer, preferably the uppermost layer of the epidermis (Stratum corneum), is (partly) dissolved, such that, firstly, fewer skin residues, if any, are available for breakdown by microorganisms such as bacteria and that, secondly, the local effect of the tyrothricin active ingredient on the surface or part of the skin from which a body odor perceived as unpleasant emanates is facilitated, promoted and/or actually enabled.

In the aforementioned composition of the invention, the keratolytic is preferably selected from the group consisting of α-hydroxy acids, preferably glycolic acid, mandelic acid and/or lactic acid; acitretin; adapalene; allantoin; aluminum oxide; azelaic acid; benzoyl peroxide; urea; isotretinoin; monochloroacetic acid; motretinide; retinoids; salicylic acid; shale oils; selenium disulfide; tazarotene; tars; tretinoin and mixtures of the aforementioned keratolytics. According to the invention, particular preference is given to urea as keratolytic.

Preference is given to a composition of the invention or to a composition of the invention specified above or hereinafter as preferred in which tyrothricin is present in an amount within a range from 0.01% to 0.5% by weight, preferably within a range from 0.05% to 0.2% by weight, based on the total mass of the composition,
and/or (preferably “and”)
in which urea is present in an amount within a range from 0.5% to 20.0% by weight, preferably within a range from 1.0% to 15.0% by weight, more preferably within a range from 3.0% to 12.0% by weight, based on the total mass of the composition.

Preference is given to a composition of the invention or to a composition of the invention specified above or hereinafter as preferred, further comprising one or more polyethylene glycols (also referred to hereinafter as “PEGs”) and/or propylene glycol. Such compositions of the invention may advantageously—especially when dispensing with other constituents—be odor-neutral (odorless); particular preference is given to such a composition.

Preferred polyethylene glycols for use in compositions of the invention or preferred compositions of the invention are selected from the group of the polyethylene glycols having an average relative molecular mass in the range from 200 to 12 000, preferably in the range from 200 to 8000, most preferably in the range from 200 to 6000, preferably determined by means of size exclusion chromatography. Particularly preferred types of polyethylene glycols are selected from the group consisting of PEG 300, PEG 400, PEG 600, PEG 1500, PEG 2000, PEG 3000 and PEG 4000, where the numbers, in a manner customary to the person skilled in the art, each state the average relative molecular masses. It is possible to use one polyethylene glycol type of a particular average relative molecular mass, e.g. “PEG 300” or “PEG 1500”, on its own, or it is advantageously possible to use mixtures of two or more polyethylene glycol types, each of a particular average relative molecular mass, e.g. “PEG 300” in a mixture with “PEG 1500”. By the selection of polyethylene glycol types of suitable average relative molecular masses or of mixtures of two or more polyethylene glycol types each of a particular average relative molecular mass, it is possible to produce compositions of the invention of different consistencies or viscosities and produce them such that they are matched to the desired end use (for instance as an ointment or as a filling for a roll-on deodorant).

For the purposes of the present invention, propylene glycol (1,2-dihydroxypropane) includes all isomers of this compound, both the optically active compounds ((R)- and(S)-1,2-dihydroxypropane) and mixtures thereof, especially the racemate.

Preference is given to a composition of the invention or to a composition of the invention specified above or hereinafter as preferred that contains polyethylene glycols and/or propylene glycol (as described above, in each case individually or as mixtures of multiple polyethylene glycol types with one another and/or with propylene glycol) in a total amount (polyethylene glycol(s) plus propylene glycol) in the range from 15% to 90% by weight, preferably in the range from 30% to 90% by weight, more preferably in the range from 40% to 90% by weight, based on the total mass of the composition.

The above-described preferred compositions of the invention comprising one or more polyethylene glycols and/or propylene glycol have a number of advantages:

For instance, aforementioned compositions of the invention containing a total amount of 15% by weight or more, based on the the total weight (or the total mass) of the composition, of one or more polyethylene glycols and/or propylene glycol can have an antibacterial or preservative effect, such that additional preservatives are generally unnecessary or need not be added to the compositions.

Inventive compositions of oily consistency (“oils”), to increase their stability, are preferably dispensed and stored in brown glass bottles; inventive compositions in the form of an ointment are preferably dispensed and stored in a coated, preferably PVC-free, aluminum tube.

In addition, polyethylene glycols and propylene glycol have very good compatibility.

It has also been found in in-house experiments that any possible risk of (possibly slow onset of) breakdown of urea and resultant rising pH values in the composition that could lead to impairments of the tyrothricin active ingredient did not occur or were not observed in compositions of the invention that contained polyethylene glycols and/or propylene glycol. By contrast, such aforementioned preferred compositions of the invention were stable even over prolonged periods.

A further advantage of the use of propylene glycol in the context of the present invention is a penetration-enhancing effect, which means that the above-described advantageous keratolytic effect of urea can be promoted or enhanced.

Particular preference is therefore given to a composition of the invention or to a composition of the invention specified above or hereinafter as preferred, comprising, in addition to tyrothricin and urea, solely polyethylene glycols and/or propylene glycol.

In many cases, preference is also given to a composition of the invention or to a composition of the invention specified above or hereinafter as preferred that additionally contains a wetting agent, preferably selected from the group consisting of polydimethylsiloxanes, polyethylene oxides, polysorbates and mixtures thereof. A particularly preferred wetting agent in this connection is polydimethylsiloxane (PDMS), also known by the name dimeticone (INN), CAS RN 9006-65-9.

Likewise preferred in many cases is a composition of the invention or a composition of the invention specified above or hereinafter as preferred that additionally contains a skin-compatible pH regulator, preferably a buffer system, more preferably a buffer system suitable for adjusting or buffering a pH in the range from 7.1 to 8.5, preferably in the range from 7.5 to 8.0, for example the following buffers that are known per se: phosphate buffer, tris(hydroxymethyl)aminomethane buffer (also referred to as trometamol) or borate buffer. A composition of the invention containing urea as keratolytic preferably has a pH of not higher than 8.0 which is preferably stabilized by a buffer system.

Preference is likewise given to a composition of the invention or to a composition of the invention specified above or hereinafter as preferred that additionally contains a denaturing agent, preferably a denatonium derivative, more preferably selected from the group consisting of denatonium benzoate and denatonium saccharinate.

The composition of the invention or the composition of the invention specified above or hereinafter as preferred is preferably a topical composition, more preferably in a form selected from the group consisting of cream, gel, lotion, powder, powder spray, oil, roll-on formulation, ointment, foam, spray, stick and tincture, more preferably from the group consisting of cream, gel, lotion, powder, powder spray, oil, roll-on formulation, ointment and spray.

For the use or application on the foot which is preferred in accordance with the invention, including for use for the purpose of recurrent prophylaxis, preference is given to a topical composition of the invention in a form selected from the group consisting of cream, powder, powder spray, ointment and spray. In this connection, particular preference is given to a topical composition of the invention in the form of a cream, ointment or powder. Most preferred in this connection is a topical composition of the invention in the form of a cream or ointment.

For the use or application in or beneath the armpit which is preferred in accordance with the invention, including for use for the purpose of recurrent prophylaxis, preference is given to a topical composition of the invention in a form selected from the group consisting of gel, lotion, powder, oil, roll-on formulation, foam, spray and stick. In this connection, particular preference is given to a topical composition of the invention in the form of a cream, ointment, oil or roll-on formulation.

For use or application on or to the mucosa, preference is given to a topical composition in a form selected from the group consisting of cream, gel, lotion, powder, powder spray, ointment, foam, spray, stick and tincture. In this connection, particular preference is given to a topical composition in the form of a cream, a gel, an ointment, a foam or a spray.

The invention also relates to the use of a composition of the invention or of a composition of the invention specified above as preferred and/or of a topical composition of the invention or a topical composition of the invention specified above as preferred, in each case for treatment and/or prophylaxis of human body odor, preferably in the treatment and/or prophylaxis of human body odor, where the body odor emanates from the surface of the skin, preferably from the foot and/or the armpit and/or an intertriginous region.

All the configurations and combinations of the invention that have been specified above for the inventive use of tyrothricin, including the configurations and combinations specified as preferred in each case, also apply without restriction and if appropriate mutatis mutandis to the above-specified use of a composition of the invention or preferred composition of the invention and/or of a topical composition, in each case for treatment and/or prophylaxis of human body odor.

The invention likewise relates to a composition of the invention or composition of the invention specified above as preferred and/or a topical composition of the invention or topical composition of the invention specified above as preferred, in each case for use in the treatment and/or prophylaxis of human body odor.

All configurations and combinations of the invention that have been specified above for the inventive use of tyrothricin, including the configurations and combinations specified as preferred in each case, also apply without restriction and if appropriate mutatis mutandis to the above-described composition and/or topical composition, in each case for use in the treatment and/or prophylaxis of human body odor.

The use or application of the composition of the invention or of a composition of the invention specified as preferred above, comprising tyrothricin and a keratolytic, preferably urea, is preferably on the surface of the skin, preferably at sites where the skin or horny skin is poorly ventilated and/or at sites where the skin or horny skin is swollen and/or wholly or partly macerated, has lesions (particularly in cases of “Keratoma sulcatum”, “pitted keratolysis” or “plantar pitting”, see above) and/or has symptoms of bromhidrosis, especially bromhyperhidrosis, apocrine bromhidrosis, eccrine bromhidrosis and/or keratogenic eccrine bromhidrosis. Particular preference is therefore given to the use or application of the composition of the invention or of a preferred composition of the invention at the surface of the skin of the foot, especially the sole of the foot and/or the spaces between the toes, on the hand surfaces and/or in intertriginous regions, most preferably on the surface of the skin of the foot, especially the sole of the foot and/or the spaces between the toes.

The present invention also relates to a kit comprising the following spatially separated constituents:
a) a composition suitable for topical administration, comprising tyrothricin in an antimicrobially effective amount, and
b) at least one composition suitable for topical administration, in each case containing at least one keratolytic in a keratolytically effective amount.

A “composition suitable for topical administration” is preferably selected from the group consisting of cream, gel, lotion, powder, powder spray, roll-on formulation, ointment, foam, spray, stick and tincture, more preferably from the group consisting of cream, gel, lotion, powder, powder spray, roll-on formulation, ointment and spray. In the case of the kit of the invention, the term “composition suitable for topical administration” also includes premixes and precursors that first have to be converted to a ready-to-use form or to a ready-to-use composition prior to use or application as intended.

In one embodiment, the invention also relates to an above-specified kit for use in the treatment and/or prophylaxis of human body odor and/or the use of an above-specified kit of the invention for treatment and/or prophylaxis of human body odor.

In a further embodiment, the invention also relates to a kit for the above-specified application or use in the treatment and/or prophylaxis of human body odor, wherein the application comprises the preferably alternating application of components a) and b) to the wound-free skin over a treatment period sufficient to reduce the body odor associated with perspiration at least for a period of success, and preferably to eliminate it at least for a period of success. The terms “treatment period” and “period of success” in the context of the present invention preferably have the same meaning as already specified above (in connection with the description of a successful application).

The present invention further relates to a footwear article, preferably shoe insert, insole, stocking, sock or sockliner, comprising an antimicrobially effective amount of tyrothricin.

Preferably, in all aspects of the invention (use, application, composition, use/application of the composition; kit, use of the kit), aside from tyrothricin, no further active antibacterial ingredient (antibiotic) or antiseptic ingredient is used, although a keratolytic usable in accordance with the invention, preferably urea, should not be regarded as an active antibacterial and/or antiseptic ingredient that should preferably be avoided in the context of the present invention. One advantage with which the use of tyrothricin as the sole active antibacterial and/or antiseptic ingredient is associated is that an inventive treatment of body odor brings only a very small risk of the evolution or formation of resistant germs.

### EXAMPLES

The examples specified hereinafter are intended to describe the invention in detail without limiting its scope.

### Example 1: Production of a Composition (Ointment) of the Invention, Comprising Tyrothricin and a Keratolytic

The ingredients/constituents specified below in table 1 were combined and mixed with one another in a manner known per se to give an ointment of homogeneous appearance that had an odor-neutral appearance.

TABLE 1
Composition of an ointment of the invention

| Ingredient | Amount [g] |
|---|---|
| Tyrothricin | 0.1 |
| Urea (as keratolytic) | 10.0 |
| PEG 300 | 43.2 |
| PEG 1500 | 46.7 |

The composition (ointment) of the invention produced in example 1 is particularly suitable for application to the skin of the foot affected by body odor, especially the soles of the feet.

### Example 2: Production of a Composition (Oil) of the Invention, Comprising Tyrothricin and a Keratolytic

The ingredients/constituents specified below in table 2 were combined and mixed with one another in a manner known per se to give an oil of homogeneous appearance that had a colorless and odor-neutral appearance.

TABLE 2
Composition of an oil of the invention

| Ingredient | Amount [g] |
|---|---|
| Tyrothricin | 0.1 |
| Urea (as keratolytic) | 10.0 |
| PEG 300 | 89.9 |

The composition (oil) of the invention produced in example 2 is suitable, for example, for filling of a roll-on deodorant or for direct application to parts of the skin affected by body odor, for instance to the soles of the feet or in axillary or intertriginous regions.

### Example 3: Production of a Composition (Oil) of the Invention, Comprising Tyrothricin and a Keratolytic

The ingredients/constituents specified below in table 3 were combined and mixed with one another in a manner known per se to give an oil of homogeneous appearance that had a colorless and odor-neutral appearance.

TABLE 3
Composition of a further oil of the invention

| Ingredient | Amount [g] |
|---|---|
| Tyrothricin | 0.1 |
| Urea (as keratolytic) | 10.0 |
| Propylene glycol (PH. EUR. 8.0) | 89.9 |

The composition (oil) of the invention produced in example 3 is suitable, for example, for filling of a roll-on deodorant or for direct application to parts of the skin affected by body odor, for instance to the soles of the feet or in axillary or intertriginous regions.

### Example 4: Test Series for Verifying the Efficacy of Tyrothricin in the Treatment of Body Odor on Test Subjects

A test series was conducted with 22 volunteers (“test subjects”; 14 male, 8 female) who, before the start of the test series, suffered from body odor perceived as unpleasant that emanated from the surface of the skin of the foot (“foot odor”).

To assess the intensity of the foot odor of the test subjects, the test manager employed a subjective scale of odor assessment by which the test subjects were classified into the following groups shown in table 4:

TABLE 4
Division of the test subjects according to intensity of foot odor

| Scale value | Intensity of foot odor | Number of test subjects |
|---|---|---|
| 0 | Neutral | No treatment needed |
| 1 | Barely noticeable | Exclusion criterion |
| 2 | Noticeable | 1 |
| 3 | Distinctly perceptible | 7 |
| 4 | Strong | 8 |
| 5 | Very strong | 6 |

Inclusion criteria for participation in the test series:
foot odor of at least intensity 2 on subjective scale of table 4
intact, non-pathologically altered skin of the foot or soles of the feet (no surface wounds or injuries).

Exclusion criteria for participation in the test series:
pathological alterations to the skin of the foot (back of the foot, sole of the foot, spaces between the toes), such as open wounds, allergic or idiopathic dermatoses, ulcers etc.; hornification, warts or onychomycoses were not exclusion criteria.

Procedure for the test series:
Over a period of 6 days in each case (“treatment period”), the test subjects were administered once daily with a tyrothricin-containing composition (“test ointment”, containing 0.1% tyrothricin and in accordance with the composition of example 1) over the full area of the ridged skin and about 5 mm beyond the boundary to the cutis on the right foot (the left foot served as control), including the toes and spaces between the toes. The toenails were excluded from the application and were cut back as far as possible prior to commencement of the test series. After the application of the test ointment, the feet of the test subjects were not washed for at least 12 hours.

The test subjects were to not change their personal care habits during the duration of the test series and for a subsequent period after the end of the test series (30 days), but were to stop any individual measures or means of treatment of foot odor (other than the treatment of the invention) for the duration of the test series and/or the subsequent period. The wearing of shoes was allowed, but walking barefoot was to be avoided as far as possible.

On each of the 6 treatment days, prior to application of the test ointment, the test manager assessed and rated the intensity of foot odor (by the scale in table 2). Before a new application of the test ointment, it was ensured in each case that no unwanted or pathological skin alterations had occurred as a result of or during the treatment.

### Test Results:

Immediately after completion of the treatment period, the following results were achieved (scale values for the intensity of foot odor according to table 4):
In the case of the test subject with foot odor of scale value 2, barely any foot odor perceived as unpleasant was perceptible on the 2^{nd} day of treatment.

In the case of the test subjects with foot odor of scale values 3 to 5, a significant decline in foot odor (that may have been) perceived as unpleasant by 2 to 3 scale values in each case was recorded from the 3^{rd} day of treatment.

From the fifth day of treatment, more significant foot odor than corresponded to scale value 2 was not perceived in any of the test subjects. For 15 test subjects, the intensity of the foot odor was assessed with scale values of 0 or 1 immediately after the end of the treatment period.

No unwanted side-effects (such as skin irritation or undesirable changes in odor) occurred in any of the test subjects.

The treatment with the test ointment was successful for all test subjects; no treatment failures were identified.

After conclusion of the treatment period, the test subjects were surveyed weekly as to the duration of treatment success over a subsequent period of 28 days. The result of these weekly surveys was that the positive treatment result still lasted even 14 days after conclusion of the treatment for 66% of the test subjects. For 50% of the test subjects, the positive treatment result still lasted even 21 days after conclusion of the treatment.

The recorded outcome of the aforementioned test series is thus that the treatment of body odor perceived as unpleasant with tyrothricin occurs rapidly, lasts over a prolonged period (generally ≥14 days), is highly likely to be successful (no therapy failures were identified), and is well tolerated (no side-effects were found).

1. A method for treatment and/or prophylaxis of human body odor, comprising:
applying tyrothricin or a composition comprising tyrothricin to a foot of a human.

2. The method as claimed in claim 1, wherein the applying step comprises applying tyrothricin or a composition comprising tyrothricin to intertriginous regions of the foot.

3. The method as claimed in claim 1, wherein the body odor is caused and/or promoted and/or triggered by bacterial colonization and/or bacterial metabolism products and/or perspiration and/or wherein the body odor is associated with one or more of the aforementioned phenomena.

4. The method as claimed in claim 1, wherein the application is local and superficial and/or
wherein the application is to wound-free skin.

5. The method according to claim 1, comprising applying the composition comprising tyrothricin to a foot of a human, wherein the composition comprises tyrothricin and at least one keratolytic.

6. The method as claimed in claim 5, in which the at least one keratolytic is selected from the group consisting of α-hydroxy acids; acitretin; adapalene; allantoin; aluminum oxide; azelaic acid; benzoyl peroxide; urea; isotretinoin; monochloroacetic acid; motretinide; retinoids; salicylic acid; shale oils; selenium disulfide; tazarotene; tars; tretinoin and mixtures of the aforementioned keratolytics.

7. The method as claimed in claim 5, wherein the at least one keratolytic comprises urea.

8. The composition as claimed in claim 5, wherein
tyrothricin is present in the composition in an amount within a range from 0.01% to 0.5% by weight, based on the total mass of the composition,
and/or
urea is present in the composition in an amount within a range from 0.5% to 20.0% by weight, based on the total mass of the composition.

9. The method as claimed in claim 5, wherein
tyrothricin is present in the composition in an amount within a range from 0.05% to 0.2% by weight, based on the total mass of the composition,
and/or
urea is present in the composition in an amount within a range from 1.0% to 15.0% by weight, based on the total mass of the composition.

10. The method as claimed in claim 5, wherein the composition further comprises polyethylene glycol and/or propylene glycol.

11. The method as claimed in claim 5, wherein the composition is a topical composition in a form selected from the group consisting of cream, gel, lotion, powder, powder spray, oil, roll-on formulation, ointment, foam, spray, stick and tincture.

12. The method as claimed in claim 1, wherein the method is a nontherapeutic treatment and/or prophylaxis of human body odor.

13. The method as claimed in claim 1, wherein the method is a therapeutic treatment and/or prophylaxis of human body odor.

14. The method as claimed in claim 1, further comprising applying the tyrothricin or the composition comprising tyrothricin to a region selected from the group consisting of an armpit and an intertriginous region of a foot.

15. The method as claimed in claim 14, wherein the application is local and to wound-free skin.

16. The method as claimed in claim 5, wherein the tyrothricin is present in the composition in an antimicrobially effective of amount, and the at least one keratolytic is present in the composition in a keratolytically effective amount.

17. The method as claimed in claim 5, wherein urea is present in the composition in an amount within a range from 3.0% to 12.0% by weight, based on the total mass of the composition.

18. The method as claimed in claim 5, wherein the composition comprises the tyrothricin and the at least one keratolytic in an amount effective for treatment and/or prophylaxis of human body odor.

19. The method as claimed in claim 5, wherein the composition further comprises polyethylene glycol and/or propylene glycol in a total amount in the range from 40% to 90% by weight, based on the total mass of the composition.

20. The method as claimed in claim 5, wherein, aside from tyrothricin, no further active antibacterial ingredient, antibiotic or antiseptic ingredient is present in the composition.
